(19) United States
(12) Patent Application Publication
(10) Pub. No.: US 2004/0235727 A1
(43) Pub. Date: Nov. 25, 2004

(54) Pharmaceutical formulation

(76) Inventors: Susanna Abrahmsen Alami, Molndal (SE); Anette Larsson, Molndal (SE); Jan-Erik Lofroth, Molndal (SE); Adam Rosinski, Molndal (SE)

Correspondence Address: ROPES & GRAY LLP, ONE INTERNATIONAL PLACE, BOSTON, MA, 02110-2624, US

(21) Appl. No.: 10/490,072

(22) PCT Filed: Oct. 8, 2002
(86) PCT No.: PCT/SE02/01827

(30) Foreign Application Priority Data
Oct. 9, 2001 (SE) 0103369-5

(51) Int. Cl. A61K 38/17; A61K 9/14

(52) U.S. Cl. 514/12; 424/486

(57) ABSTRACT

The present invention provides, for example, a pharmaceutical formulation for oral administration comprising H 376/95, sodium dodecyl sulphate (SDS) and a polymer selected from the group comprising hydroxypropyl methylcellulose (HPMC), hydroxyethyl cellulose (HEC) and polyoxyethylene oxide (PEO), a process for the preparation of such a formulation and the medical use of the formulation in the treatment of thromboembolism.

The present invention relates to a new pharmaceutical formulation for oral administration comprising a basic, low molecular weight thrombin inhibitor. The invention also relates to a process for the preparation of such a formulation and to the medical use of the formulation in the treatment of thromboembolism.

A controlled release pharmaceutical composition is disclosed in EP-A1-0214735.

Basic, low molecular weight peptide thrombin inhibitors (such as melgatran, inogatran and H 376/95 {EtO₂C—CH₂—RCgl-Aze-Pab-OH; see Example 17 of WO97/23499; Glycine, N-[1-cyclohexyl-2-[2-[[[[4-[(hydroxyimino)aminomethyl]-phenyl]methyl]amino]carbonyl]-1-azetidinyl]-2-oxoethyl]-, ethyl ester, [S—(R*, S*)]-}) are effective for the treatment of a number of diseases characterised by hypercoagulation. They are characterised by a low solubility at basic pH where the molecule is uncharged. The solubility is dramatically increased in the protonated form at acidic pH. The change in pH along the gastrointestinal tract (GI tract) causes a basic drug, when administered orally, to show variable dissolution rates and saturation concentrations at the different pHs. Thus, upon administration of a conventional formulation (for example an HPMC matrix), fast release of the drug is obtained in the stomach (low pH), while markedly slower release is obtained in the intestine (neutral pH). This variability in release behaviour of a basic drug is not acceptable as a safe, efficient and convenient therapy. The compound H 376/95 can be prepared in a number of different crystalline forms (see U.S. Pat. No. 6,225,287 and WO00/14110).

In recent years there has been a large increase in the development and use of so called modified release (MR) tablets. A modified release dosage formulation is one in which the formulation is adjusted such that drug release characteristics of time after ingestion and/or location in the GI tract are obtained to accomplish therapeutic or convenience objectives not offered by conventional dosage forms such as solutions, ointments or promptly dissolving dosage forms. There are different formulation principles to obtain modified release of a drug, (see, for example, Langer and Wise (Eds.) “Medical applications of controlled release”, vols. I and II, CRC Press Inc, Boca Raton, 1984; Robinson and Lee (Eds.) “Controlled drug delivery—fundamentals and applications”, Marcel Dekker, NY, 1987; Bogentoft and Sjögren “Towards better safety of drugs and pharmaceutical products” (Ed.: Braimer), Elsevier, 1980). In general modified release is obtained according to one, or a combination of more than one, of the principles listed below. The actual approach chosen for a given drug depends, inter alia, on the physical properties of that drug. The principles are:

i. Formulating a drug in an insoluble matrix so that the swelling kinetics of the swelling matrix, the dissolution rate of the drug, and the diffusion of the drug through the matrix all contribute to the overall release rate. The same principle applies when drug particles or cores containing the drug are coated with an insoluble but porous membrane of polymers.

ii. Formulating a drug in an eroding matrix of a soluble polymer the release rate of the drug will depend on both the swelling and the erosion rates of the matrix, and the dissolution and diffusion rates of the drug.

iii. Placing a semipermeable membrane around a tablet or drug particle, which membrane allows water in (by osmosis), the water dissolves drug and drug solution is discharged through an orifice in the membrane as a result of increased internal pressure. The size of the orifice in the membrane controls both the water flow through the membrane, and the release rate of drug solution.

Ordinary modified release formulations are often not suited to active substances that show pH dependent dissolution (G. S. Banker, Medical Application of Controlled Release (Eds. Langer and Wise), CRC Press Inc, Boca Raton, 1984; p1, vol. II). In all the formulations discussed above drugs with pH dependent solubility would give rise to unpredictable, uncontrolled and unacceptable release characteristics.

In the literature several means to achieve pH independent release of active substances have been described. Kohri et al. (Int. J. Pharmaceutics 68 225 (1991)) used the concept of introducing citric acid into a polymer matrix of PVP (polyvinylpyrrolidone) creating a microclimate of suitable pH in the matrix. However, the diffusion of such added substances out of the matrix could be too high to keep the intended micro pH for a substantial time.

Alternatively, polymer having different properties can be combined so that both an enhanced and decreased effect on the dissolution rate can be obtained (Kohri et al. Int. J. Pharmaceutics 81, 4 (1992); Giunchedi P. et al. Int. J. Pharmaceutics 85, 141 (1992)). Feely et al. (Int. J. Pharmaceutics 44, 131 (1988)) showed that incorporation of charged polymer into a non-ionic polymer matrix could result in a decreased dissolution rate of an active substance if the active substance had opposite charge compared to the added charged polymer.

Smith and Macrae have disclosed (Proc 2^{nd} World Meeting APGI/APV, Paris 1998, p 325) a hydrophilic matrix of HPMC and low molecular weight PEO that have an erosion rate that increases with time. This formulation can be used to obtain pH independent release of weakly basic drugs when passing through the GI tract.

The effect of surface active agents in hydrophilic matrixes can be many fold. Aggregates formed by the surfactants are highly charged and function from this point of view in the same way as a charged polymer. Since diffusion of micelles in a polymer matrix is highly reduced it is expected that the electrostatically “bound” or solubilised drug would diffuse slowly as well, leading to a retarded release. Further, during dissolution of a surface active agent, several different more or less slowly dissolving liquid crystalline phases of the agent might be necessary to pass. This is determined by the phase diagram of the agent and is an inherent property of surface active agents. Slow dissolution of such phases might then also contribute to a slower diffusion of an associated drug, with retarded release as a consequence. Finally, complexes might form between the drug/surfactant, drug polymer/surfactant, or surfactant/polymer that are more or less easily soluble.

Surface active agents are known to be best suited to achieve an increase in the solubility or the dissolution rate of substances that have low water solubility. Some authors have however also used surface active substances or lipid systems to control the release rate of water soluble drugs. Feely et al (Int. J. Pharmaceutics 41, 83 (1988)) and Ford et al (Int. J. Pharmaceutics 71, 213 (1991)) studied the erosion controlled release, and showed that surface active agents of opposite charge of the drugs could significantly retard the release of the substance from a hydrophilic polymer matrix. They postulated that complexes between drug and surfactants, with low aqueous solubility, were formed within the HPMC matrix and that the drug release then primarily will be determined by the erosion rate of the matrix. A similar concept was presented in U.S. Pat. No. 4,834,965, where a water soluble complex between drug and an anionic surfactant was formed. The release of the drug from the complex incorporated in a polymer matrix was in that patent claimed to be pH-independent. However, the release from the said matrix is not constant. In addition it was not mentioned in the patent that the used drug has a pH-dependent solubility. Surfactants have also been used in hydrophilic matrixes as pure viscosity enhancers [U.S. Pat. No. 4,540,566], or as a mean to protect active substances incorporated in crystalline polymer matrixes from hydrolysis of the environment [WO89/09066].

Independently of the mechanism of action the release of a soluble drug, for our drug this is valid at acidic pH, from a hydrophilic matrix will be retarded upon addition of a surface active agent and the release rate would then change. It was not shown in any of the prior art discussed about that it would be possible to obtain a pH-independent release with constant release rate from a formulation based on a polymer matrix containing a basic drug pH dependent dissolution rates and a surfactant.

It has now surprisingly been found that a water soluble low molecular weight peptide thrombin inhibitor, having with pH dependent solubility, can be formulated in a polymer matrix together with a surface active agent to obtain a modified release system characterised by a substantially pH independent release of the thrombin inhibitor. Moreover, it was surprisingly found that pH independent constant release rate was obtained.

A constant release rate is defined in a way that the experimental values for the amount of released substance at each investigated time do not deviate more than ±5% from line obtained by fitting experimental data obtained for the specified time period using linear regression. For 8 hour tablet this means that relative residuals for the experimental points are lower than ±5% in the time interval 0 to 8 hours (when fitted to a straight line).

Thus the present invention provides a pharmaceutical formulation for oral administration comprising H 376/95, sodium dodecyl sulphate (SDS) and a polymer selected from the group comprising hydrbxypropyl methylcellulose (HPMC), hydroxyethyl cellulose (HEC) and polyoxyethylene oxide (PEO). (Note that sodium dodecyl sulphate (SDS) is the same chemical entity as: sodium lauryl sulphate (SLS), dodecyl sodium sulphate, sodium monododecyl sulphate and sodium monolauryl sulphate (see Handbook of Pharmaceutical Excipients, 2nd edition, The Pharamcaeutical Press (1994))).

The different formulations for which this can be achieved are: (i) eroding and non-eroding matrix formulations based on hydrophilic and/or hydrophobic matrix forming excipients; (ii) diffusion and/or osmotic pressure controlled membrane coated formulations; and (iii) combinations of these principles.

The matrix and membrane coated formulations may be monolithic, such as tablets, or capsules, or in the form of multiple formulations administered in a tablet, capsule or sachets.

In one aspect the present invention provides a pharmaceutical formulation in which H 376/95 is present in the range 1-50% w/w (preferably 20-40% w/w).

HPMC is, for example, HPMC 50 cps or HPMC 6 cps. “HPMC 50 cps” possesses an apparent viscosity of 40-60 mPa.s (or 40-60 cps), when measured at 20° C. with a 2% (w/w) aqueous solution, calculated with reference to the dried substance, using a capillary viscometer (Ubbelohde or equivalent). “HPMC 6 cps” possesses an apparent viscosity of 4.8-7.2 mPa.s (or 4.8-7.2 cps), when measured at 20° C. with a 2% (w/w) aqueous solution, calculated with reference to the dried substance, using a capillary viscometer (Ubbelohde or equivalent). Other HPMC grades could include, for example, “HPMC 10000 cps” or “HPMC 15000 cps”; having, respectively, apparent viscosities of 7500-14000 mPa.s (or 7500-14000 cps), and 11250-21000 mPa.s (or 11250-21000 cps)) when measured at 20° C. with a 2% (w/w) aqueous solution, calculated with reference to the dried substance, using a capillary viscometer (Ubbelohde or equivalent).

HEC is, for example, BEC “Natrosol 250 Pharma, type G” from Hercules Incorporated (Aqualon), showing a typical Brookfield viscosity of 500 mPa.s (max) using a Brookfield Synchro Lectric Model LVF instrument, at the conditions 2% solution concentration, spindle no. 2, spindle speed 60 rpm, factor 5, 25° C.; or, HEC “Natrosol 250 Pharma, type M”, showing a typical Brookfield viscosity of 10,000 mPa.s (max), using the same instrument, at the conditions 2% solution concentration, spindle no. 4, spindle speed 60 rpm, factor 100, 25° C. Other BEC grades may include, for example, HEC, “Natrosol 250 Pharma, type HH” showing a typical Brookfield viscosity of 20,000 mPas (max), using the same instrument, at the conditions 1% solution concentration, spindle no. 4, spindle speed 30 rpm, factor 200, 25° C.

PEO has, for example, a MW of ≧400,000 {corresponding to an aqueous solution viscosity of 2250-3350 cps; measured for a 5% aqueous solution at 25° C., using a Brookfield RVF voscometer with no. 1 spindle, at 2 rpm}, especially a MW of >900,000 {corresponding to an aqueous solution viscosity of 8800-17600 cps; measured for a 5% aqueous solution at 25° C., using a Brookfield RVF voscometer, with no. 2 spindle, at 2 rpm}. Other grades of PEO may include a MW of >4 million (4M) {corresponding to an aqueous solution viscosity range of 1650-5500 cps; measured for a 1% aqueous solution at 25° C., using a Brookfield RVF viscometer, with No. 2 spindle, at 2 rpm}; for example a PEO of MW around 5 million (5M) {corresponding to an aqueous solution viscosity range of 5500-7500 cps,} or around 8 million (8M) {corresponding to an aqueous solution viscosity range of 10000-15000 cps}.

In another aspect the present invention provides a pharmaceutical formulation in which HPMC, HEC or PEO is present in the range 10-80% w/w (preferably 15-70% w/w).

In a further aspect the present invention provides a pharmaceutical formulation in which the amount of polymer (for example HPMC, HEC or PEO) present is such as to provide a weight ratio of polymer: H 376/95 in the range 3:1 to 1:4 (for example in the range 2:1 to 1:3, such as about 3:2 or about 1:1). In a further aspect the present invention provides a pharmaceutical formulation in which the amount of SDS present is such as to provide a molar ratio of SDS:H 376/95 in the range 3:1 to 1:4 (for example in the range 1:1 to 1:3, such as about 2:3 or about 1:1). The amount of SDS to be used in the formulation can be optimised by deciding the desired release rate of H 376/95 from the formulation and then experimenting with different amounts of SDS to achieve a formulation having the desired release rate. A preferred ratio of SDS:H 376/95 is 2:1 based on a charge basis, or a ratio as illustrated in any of the Examples. In one aspect of the present invention one polymer is used, selected from the group comprising hydroxypropyl methylcellulose (HPMC), hydroxyethyl cellulose (HEC) and polyoxyethylene oxide (PEO).

In a further aspect of the invention, mixtures of polymers may be utilised, for example a mixture between two or three of the polymers HPMC, HEC and PEO. Within such mixtures a range of different concentrations of each polymer is possible, for example, from 1% w/w of one polymer to 50%-50% mixtures of two polymers, or equal mixtures of all three polymers. The overall concentration of polymer used in the formulations of the invention described herein apply when a single polymer is used, or when a mixture of polymers is used.

The formulation of the present invention may also include a buffer (for example a phosphate buffer, such as monobasic sodium phosphate (NaH₂PO₄), dibasic sodium phosphate (Na₂HPO₄) or citric acid monohydrate). The buffer can be ground to a very small average particle size before it used in a process to prepare a pharmaceutical formulation of the present invention.

The pharmaceutical formulation of the present invention may also comprise a filler. Suitable fillers include, for example, mannitol, microcrystalline cellulose or dicalcium phosphate.

The pharmaceutical formulation of the present invention may also comprise a binder which is soluble in a suitable solvent (such as water or ethanol). Suitable binders include polyvinylpyrrolidone (PVP).

The pharmaceutical formulation of the present invention may also comprise a stabiliser. Suitable stabilisers include propyl gallate, butylated hydroxytoluene (BHT) or vitamin E.

When the pharmaceutical formulation of the present invention is in the form of a tablet it is preferred that the formulation additionally comprises a lubricant. Suitable lubricants include PRUV™, sodium stearyl fumarate, magnesium stearate and talc. It is preferred that when present, the lubricant is present in the range 0.5-1.5% w/w.

The pharmaceutical formulation of the present invention may also comprise one or more excipients. Suitable excipients are, for example, a processing additive, plasticiser, colourant or surfactant.

The dosage form of the pharmaceutical formulation of the invention may be a solid, semi-solid or liquid preparation prepared by a known technique. For example, the pharmaceutical formulation can be in tablet or capsule form or in the form of a multiple formulation administered in a tablet, capsule or sachet. The formulations can be obtained by a range of known processes, for example, by granulation, compression, microencapsulation or spray coating.

The SDS can be ground to a very small average particle size before it used in a process to prepare a pharmaceutical formulation of the present invention.

A tablet formulation can be prepared, for example, by a direct compression or a wet granulation technique.

For the direct compression technique H 376/95 is thoroughly mixed with HPMC, HEC or PEO; and SDS and any additional excipient. A lubricant (such as sodium stearyl fumarate or PRUV™) is sieved and added to the H 376/95 mixture followed by further mixing. The resulting mixture is then compressed into tablets.

For the wet granulation technique H 376/95 is thoroughly mixed with HPMC, HEC or PEO; SDS; and, optionally, one or more fillers or one or more excipients. The resulting mixture is then moistened with:

a solution of a suitable binder (such as polyvinylpyrrolidone (PVP)) dissolved in a suitable solvent (such as ethanol or water); or,

a suitable solvent (such as ethanol or water); and the resulting blend granulated using a standard or modified granulation procedure (such as spray-granulation). After drying the resulting granulate (for example in an oven or a fluid bed) at a suitable temperature (such as about 50° C.) for a suitable period (such as 20-24 hours) the granulate is milled (for example dry- or wet-milled), mixed with a lubricant (such as PRUV™, sodium stearyl fumarate, magnesium stearate or talc) and the resulting composition compressed into tablets. The dried granulate could also be used to fill capsules (such as capsules made of gelatin).

In another aspect the present invention provides a process for preparing a pharmaceutical formulation of the invention as hereinbefore described. For example said process comprises mixing H 376/95, SDS and polymer (the polymer being selected from the group comprising HPMC, HEC and PEO) to form a powder mixture; and compressing the powder mixture to form one or more tablets.

In a further aspect the present invention provides a pharmaceutical formulation as hereinbefore described for use in therapy (such as, as a medicament for cardiovascular disorders, for example thromboembolism).

In a further aspect the present invention provides the use of a pharmaceutical formulation as described above in the manufacture of a medicament for the prophylaxis and/or treatment of a cardiovascular disorder (for example thromboembolism).

In another aspect the present invention provides a method for prophylaxis and/or treatment of a cardiovascular disorder (for example thromboembolism) which comprises the administration of a therapeutically effective amount of a pharmaceutical formulation as hereinbefore described to a mammal (such as man) in the need of such treatment.

Examples 2, 3, 5, 7 and 8 illustrate the invention. Examples 1, 4 and 6 are included for comparative purposes only. In the accompanying Figures:

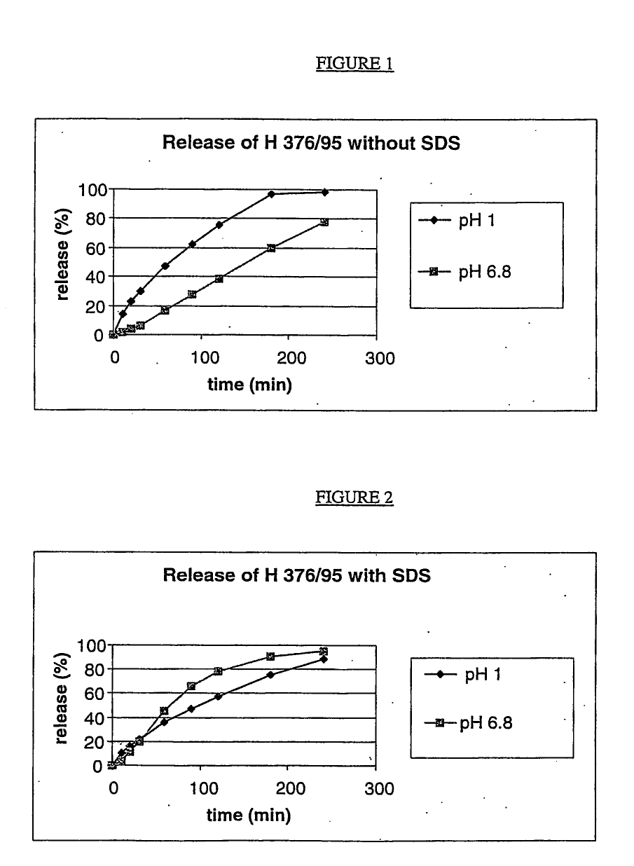

FIG. 1: Comparison between the release performance of H 376/95 at pH 1 and pH 6.8 with no SDS in the formulation.

FIG. 2: Comparison between the release performance of H 376/95 at pH 1 and pH 6.8 with SDS and buffer in the formulation.

In Examples 1 to 7 the tablets prepared were analysed for H 376/95 release in 500 ml 0.1M HCl for 2 hours followed by test in 900 ml phosphate buffer pH 6.8 (ionic strength (I) of 0.1) using a USP dissolution apparatus No. 2 (paddle+basket), 50 rpm to mimic pH variations in vivo. The amount of H 376/95 released was determined by UV-spectrometry.

### EXAMPLE 1

Example 1 demonstrates the release of H 376/95 from a hydroxypropyl methylcellulose matrix in the absence of SDS.

| | |
|---|---|
| Crystalline H 376/95 | 53.5 mg |
| Hydroxypropyl methylcellulose 50 cps | 105 mg |
| Sodium stearyl fumarate | 1.5 mg |
| Tablet weight | 160 mg |

Crystalline H 376/95 was mixed manually with hydroxypropyl methylcellulose. The powder mixture was sieved and mixed with the lubricant, sodium stearyl fumarate. The final mixture was compressed to form tablets using a single-stroke tablet machine.

The results of the analysis described above are presented in Table 1. These results clearly show that at low pH H 376/95 is protonated and highly soluble and, hence, gives a rapid release, while at high pH the drug is not ionised giving a slow release due to the low solubility of the drug in this form.

### EXAMPLE 2

Example 2 demonstrates the release of H 376/95 from a hydroxypropyl methylcellulose matrix in the presence of SDS.

| | |
|---|---|
| Crystalline H 376/95 | 50.8 mg |
| Hydroxypropyl methycellulose 50 cps | 100 mg |
| Sodium stearyl fumarate | 2.2 mg |
| Sodium dodecyl sulfate | 60 mg |
| Tablet weight | 213 mg |

Crystalline H 376/95 was mixed manually with hydroxypropyl methylcellulose and sodium dedecyl sulfate. The powder mixture was sieved and mixed with the lubricant, sodium stearyl fumarate. The final mixture was compressed to form tablets using a single-stroke tablet machine.

The results of the analysis described above are presented in Table 1. Comparing these results to the results of Example 1 it can be seen that the inclusion of SDS provides substantially pH independent release of H 376/95.

### EXAMPLE 3

This Example compares the release of H 376/95 from hydroxypropyl methycellulose matrices over 3 hours.

| | |
|---|---|
| Hydroxypropyl methylcellulose matrix - no SDS: | |
| Crystalline H 376/95 | 50.2 mg |
| Hydroxypropyl methylcellulose 50 cps | 80.2 mg |
| Sodium stearyl fumarate | 1.6 mg |
| Tablet weight | 132 mg |
| Hydroxypropyl methycellulose matrix - with SDS: | |
| Crystalline H 376/95 | 50.3 mg |
| Hydroxypropyl methylcellulose 50 cps | 80.5 mg |
| Sodium stearyl fumarate | 2.0 mg |
| Sodium dodecyl sulfate | 30.2 mg |
| Tablet weight | 163 mg |

Crystalline H 376/95 was mixed manually with hydroxypropyl methylcellulose and sodium dodecyl sulfate. The powder mixture was sieved and mixed with the lubricant, sodium stearyl fumarate. The final mixture was compressed to form tablets using a single-stroke tablet machine.

From Table 2 it can be seen that a linear pH independent release is obtained for a tablet with a 3 hour release profile when sodium dodecyl sulfate is present in the formulation.

### EXAMPLE 4

This Example demonstrates the release of H 376/95 from a polyoxyethylene oxide matrix in the absence of SDS.

| | |
|---|---|
| Crystalline H 376/95 | 2.55 g |
| Polyoxyethylene oxide MW 900,000 | 5.00 g |
| Propyl gallate | 0.008 g |
| Sodium stearyl fumarate | 0.008 g |
| Tablet weight | 153 mg |

Crystalline H 376/95 was mixed with polyethylene oxide and propyl gallate. The mixture was sieved and mixed with the lubricant, sodium stearyl fumarate. The final mixture was compressed to form tablets using a single-stroke tablet machine.

The results presented in Table 3 show the pH-dependent, release of the drug from this formulation. At low pH the drug is protonized and highly soluble, hence, a rapid release is obtained. At high pH the drug is non-ionized and slow release is obtained due to the low solubility of the drug.

### EXAMPLE 5

This Example demonstrates the release of H 376/95 from a polyoxyethylene oxide matrix in the presence of SDS.

| | |
|---|---|
| Crystalline H 376/95 | 2.55 g |
| Polyoxyethelene oxide 900,000 | 5.00 g |
| Propyl gallate | 0.011 g |
| Sodium dodecyl sulfate | 3.00 g |
| Sodium stearyl fumarate | 0.11 g |
| Tablet weight | 213 mg |

Crystalline H 376/95 was mixed with polyethylene oxide, propyl gallate and sodium dodecyl sulfate. The mixture was sieved and mixed with the lubricant, sodium stearyl fumarate. The final mixture was compressed to form tablets using a single-stroke tablet machine.

The results are also presented in Table 3. Comparing these results with the results obtained in absence of SDS (Example 4) it is clearly seen that the inclusion of SDS results in a pH independent release of H 376/95.

### EXAMPLE 6

This Example demonstrates the release of H 376/95 from a hydroxyethylcellulose matrix in the absence of SDS.

| | |
|---|---|
| Crystalline H 376/95 | 0.5 g |
| Hydroxyethylcellulose (NATROSOL G) | 0.5 g |
| Sodium stearyl fumarate | 0.013 g |
| Tablet weight | 100 mg |

Crystalline H 376/95 was mixed manually with hydroxyethylcellulose. The powder mixture was sieved and mixed with the lubricant, sodium stearyl fumarate. The final mixture was compressed to form tablets using a single-stroke tablet machine.

The results presented in Table 4 clearly show an unacceptable pH dependent release of H 376/95 from this formulation. At low pH the drug is protonized and highly soluble, hence, a rapid release is obtained. At high pH the drug is non-ionized and slow release is obtained due to the low solubility of the drug.

### EXAMPLE 7

This Example demonstrates the release of H 376/95 from a hydroxyethylcellulose matrix in the presence of SDS.

| | |
|---|---|
| Crystalline H 376/95 | 0.50 g |
| Hydroxyethylcellulose (NATROSOL G) | 0.50 g |
| Sodium dodecyl sulfate | 0.30 g |
| Sodium stearyl fumarate | 0.013 g |
| Tablet weight | 132 mg |

Crystalline H 376/95 was mixed manually with hydroxyethylcellulose and sodium dodecyl sulfate The powder mixture was sieved and mixed with the lubricant, sodium stearyl fumarate. The final mixture was compressed to form tablets using a single-stroke tablet machine.

The results are presented in Table 4. It can be concluded that the inclusion of SDS results in a pH independent release of H 376/95.

### EXAMPLE 8

This Example compares the release of H 376/95 from hydroxypropyl methycellulose matrices over 4 hours.

| | per tablet |
|---|---|
| Hydroxypropyl methylcellulose matrix - no SDS: | |
| Crystalline H 376/95 | 50 mg |
| Hydroxypropyl methylcellulose 50 cps | 44 mg |
| Sodium stearyl fumarate | 4 mg |
| MCC | 3 mg |
| Mannitol | 84 mg |
| Sodium aluminium silicate | 47 mg |
| HPC (in Ethanol solution, 10% w/w) | 20 mg |
| Tablet weight | 252 mg |
| Hydroxypropyl methylcellulose matrix - with SDS: | |
| Crystalline H 376/95 | 50.5 mg |
| Hydroxypropyl methylcellulose 50 cps | 60 mg |
| SDS | 20 mg |
| Sodium stearyl fumarate | 2.5 mg |
| Mannitol | 50 mg |
| NaH₂PO₄ (buffer) | 75 mg |
| HPC (in Ethanol solution, 10% w/w) | 13 mg |
| Tablet weight | 271 mg |

The tablets were made by wet granulation. Crystalline H 376/95, hydroxypropyl methylcellulose 50 cps and mannitol were mixed manually with microcrystalline cellulose and sodium aluminium silicate or sodium lauryl sulphate and sodium dihydrogen phosphate dihydrate. Before adding sodium dihydrogen phosphate dihydrate, the buffer was milled and/or sieved to decrease the particle size of the excipient. After a physical mixture was achieved, the powder mixture was moistened with the granulation solution and mixed until it was homogeneous. If necessary more ethanol was added. After drying the granulate in a tray oven, it was sieved though a suitable sieve. The granulate was then lubricated with sodium stearyl fumarate by stirring manually. The final mixture was compressed to form tablets using a single-stroke tablet machine.

The tablets prepared were analysed for H 376/95 release in 900 ml 0.1M HCl and in 900 ml phosphate buffer pH 6.8 (I=0.1) over 4 hours using a USP dissolution apparatus No. 2 (paddle+basket), 50 rpm to mimic pH variations in vivo. The amount of H 376/95 released was determined by UV-spectrometry.

Results shown in FIGS. 1 and 2 where graphs comparing the pH dependence, or independence, of these formulations are presented. In FIG. 1 pH dependent release is observed as H 376/95 is released significantly faster at pH 1 due to its ionised (and higher solubility) state. FIG. 2 shows that the presence of SDS (and buffer) in the formulation gives less pH dependent release, especially at shorter times. SDS appears to have a retarding effect at low pH and an enhancing effect at high pH. Approximate values for the quantity of H 376/95 released after a particular time may be derived from FIGS. 1 and 2, within a margin of error of about + or −5%.

TABLE 1
Table 1 shows the cumulative release of H 376/95
from an HPMC matrix as described in Example 1,
and an HPMC matrix modified by addition of
SDS as described in Example 2.

Cumulative % released

| Time (minutes) | Example 1 | Example 2 |
|---|---|---|
| 15 | 14 | 5 |
| 30 | 24 | 9 |
| 60 | 40 | 12 |
| 90 | 53 | 16 |
| 110 | 61 | 20 |
| 150 | 68 | 34 |
| 180 | 71 | 39 |
| 240 | 76 | 48 |
| 300 | 81 | 58 |
| 360 | 86 | 68 |
| 420 | 92 | 80 |
| 480 | 97 | 93 |

TABLE 2
Table 2 shows the cumulative release, over 3 hours,
of H 376/95 from HPMC matrixes with and without
SDS described in Example 3.

Cumulative % released

| Time (minutes) | Without SDS | With SDS |
|---|---|---|
| 15 | 15 | 9 |
| 30 | 30 | 17 |
| 50 | 65 | 26 |
| 90 | 69 | 45 |
| 120 | 73 | 51 |
| 150 | 79 | 64 |
| 180 | 82 | 76 |
| 210 | 84 | 84 |
| 240 | 85 | 88 |

TABLE 3
Release of Compound A from polyoxyethylene oxide (PEO) matrix
as described in Example 4 and PEO matrixes modified by addition
of SDS as described in Example 5.

Cumulative % released

| Time (minutes) | Example 4 | Example 5 |
|---|---|---|
| 15 | 13 | 3 |
| 30 | 22 | 5 |
| 60 | 38 | 10 |
| 90 | 53 | 15 |
| 110 | 61 | 20 |
| 150 | 71 | 35 |
| 180 | 76 | 45 |
| 240 | 83 | 64 |
| 300 | 89 | 78 |
| 360 | 96 | 86 |
| 420 | 99 | 92 |
| 480 | 100 | 98 |

TABLE 4
Release of Compound A from hydroxyethylcellulose (HEC) matrix
as described in Example 6 and HEC matrix modified by addition
of SDS as described in Example 7.

Cumulative % released

| Time (minutes) | Example 6 | Example 7 |
|---|---|---|
| 15 | 21 | 7 |
| 30 | 32 | 13 |
| 60 | 50 | 23 |
| 90 | 66 | 32 |
| 110 | 74 | 37 |
| 150 | 81 | 52 |
| 180 | 84 | 60 |
| 240 | 88 | 71 |
| 300 | 92 | 79 |
| 360 | 94 | 86 |
| 420 | 96 | 90 |
| 480 | 97 | 92 |

1. A pharmaceutical formulation for oral administration comprising H 376/95, sodium dodecyl sulphate (SDS) and a polymer selected from the group comprising hydroxypropyl methylcellulose (HPMC), hydroxyethyl cellulose (HEC) and polyoxyethylene oxide (PEO).

2. A pharmaceutical formulation as claimed in claim 1 wherein the polymer is hydroxypropyl methylcellulose (HPMC).

3. A pharmaceutical formulation as claimed in claim 1 wherein the polymer is hydroxyethyl cellulose (HEC).

4. A pharmaceutical formulation as claimed in claim 1 wherein the polymer is polyoxyethylene oxide (PEO).

5. A pharmaceutical formulation as claimed in claim 1 for use in therapy.

6. The use of a pharmaceutical formulation as claimed in claim 1 in the manufacture of a medicament for the prophylaxis and/or treatment of a cardiovascular disorder.

7. A method for prophylaxis and/or treatment of a cardiovascular disorder which comprises the administration of a therapeutically effective amount of a pharmaceutical formulation as claimed in claim 1 to a mammal in the need of such treatment.

8. A process for the preparation of a pharmaceutical formulation as claimed in claim 1 which comprises mixing H 376/95, sodium dodecyl sulphate (SDS) and polymer (the polymer being selected from the group comprising hydroxypropyl methylcellulose (HPMC), hydroxyethyl cellulose (HEC) and polyoxyethylene oxide (PEO)) to form a powder mixture; and compressing the powder mixture to form one or more tablets.

9. A process for the preparation of a pharmaceutical formulation as claimed in claim 1 which comprises:
1. mixing H 376/95, sodium dodecyl sulphate (SDS), polymer (the polymer being selected from the group comprising hydroxypropyl methylcellulose (HPMC), hydroxyethyl cellulose (HEC) and polyoxyethylene oxide (PEO)) and, optionally, one or more fillers or one or more excipients;
2. moistening the resulting mixture with:
a. a solution of a suitable binder dissolved in a suitable solvent; or,
b. a suitable solvent; and;
3. granulating the resulting blend.
